(19) United States
(12) Patent Application Publication
(10) Pub. No.: US 2005/0050587 A1
(43) Pub. Date: Mar. 3, 2005

(54) Inducers of plant disease resistance

(76) Inventors: F. Paul Silverman, Highland Park, IL (US); Peter D. Petracek, Grayslake, IL (US); Daniel F. Heiman, Libertyville, IL (US); Christina M. Fledderman, Houston, TX (US); Prem Warrior, Green Oaks, IL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/928,419

(22) Filed: Aug. 27, 2004

Related U.S. Application Data
(60) Provisional application No. 60/499,413, filed on Aug. 29, 2003.

(51) Int. Cl. A01H 1/00; C12N 15/82; A01N 63/00

(52) U.S. Cl. 800/280; 504/117

(57) ABSTRACT

Use of salicylates and 3-hydroxypicolinic acid to induce production of PR-1a protein, to increase resistance of plans to tobacco mosaic virus and to potentiate plant protective activity of SAR inducers.

### BACKGROUND OF THE INVENTION

Like animals, plants have evolved means of defending themselves against disease. In response to disease-causing agents, a plant may turn on a resistance response involving a complex array of defenses. These include increased mechanical barriers, biochemical defenses, and programmed cell death in the path of an invading pathogen.

The plant resistance response is often characterized by localized cell death at the site of pathogen ingress called the hypersensitive response (HR). HR restricts the spread of the pathogen, and may prevent it from causing disease. Reactive oxygen species (ROS) may be involved in the generation of HR (Kombrink et al. 2001. European Journal of Plant Pathology 107:69). Although the formation of HR is not fully understood, it is the most visible of the suite of anti-pathogen responses induced during the defense response. These include the synthesis of anti-microbial compounds known as phytoalexins, increases in phenylpropanoid metabolism, and the synthesis of new classes of host proteins.

Following HR, a generalized resistance to pathogen attack may develop in the plant. First observed in the area surrounding the HR lesion, increased resistance is subsequently observed throughout the plant, and is not pathogen specific. This phenomenon is known as systemic acquired resistance (SAR), and has been known since the late 19^{th} century (Chester, 1933. Quarterly Review of Biology 8:275). Ross (1961. Virology 14:340) further characterized SAR, and provided a scientific basis for its study. Although much of the early work on SAR was performed using the N-gene tobacco/TMV pathosystem, it has been studied in other plants including cucumber (Rasmussen et al. 1991. Plant Physiology 97:1342) Arabidopsis (Maldonado et al. 2002. Nature 419:399) and rice (Schweitzer et al. 1997. Plant Physiology 115:61).

In conjunction with HR and SAR, the synthesis of several new classes of proteins, the pathogenesis-related (PR) proteins, has been observed. During the establishment of tobacco SAR, at least six classes of PR proteins are synthesized (Van Loon 1997. European Journal of Plant Pathology 103:753). Of these, at least two classes are directly anti-microbial. The most abundant class of PR proteins, PR-1, has no known function, but when PR-1a was continuously expressed in tobacco, tolerance to oomycete pathogens was observed (Alexander et al. 1993. Proceedings of the National Academy of Science USA 90:7327). However, the relationship between PR proteins and plant resistance to disease is complex. Although the endogenous SAR signal salicylic acid (SA) acts through a PR protein-dependent pathway, another disease resistance pathway, the induced systemic resistance pathway (ISR) is independent of SA (Heil et al. 2002. Annals of Botany 89:503).

The existence of SAR postulates a signal that moves from the site of pathogen attack throughout the plant. The signal would systemically induce plant defenses. As plants lack a circulatory system capable of delivering large molecules, small molecules, either airborne (e.g. ethylene, methyl jasmonate) or through the vascular system (e.g. auxins, cytokinins) act as potent regulators of plant growth and development. A large body of evidence supports the role of SA as a signal molecule in SAR. Increases in endogenous SA levels are observed during the resistance response in many dicot plant species. Exogenously applied SA induces PR protein expression and enhanced resistance in tobacco (White, 1979. Virology 99:410). Transgenic plants expressing the nahG gene, encoding a salicylate hydroxylase that metabolizes SA to catechol, fail to develop resistance when treated with SA and are more susceptible to pathogen attack (Gaffney et al. 1993. Science 261:754). Although the primary mobile signal in SAR remains unclear, SA appears to be necessary for the establishment of SAR.

The role of active oxygen species in the resistance response has also been a matter of some debate. Increases in hydrogen peroxide have been observed as a rapid response to pathogen attack, and may function as a signal for programmed cell death (Levine et al. 1994. Cell 79:583). Other reactive oxygen species, including nitric oxide may play a role in the resistance response, and may function as local signals. (Delledonne et al. 1998. Nature 394:585). Increased levels of hydrogen peroxide have been postulated to play a role in SAR due to SA-mediated inhibition of catalase (Chen et al. 1993. Science 262:1883).

During the past decade, several products that induce SAR have been commercialized as plant protection chemicals. These products are either elicitors, which mimic an attack of a plant pathogen or are functional analogues of salicylic acid. Examples of the elicitors include Harpin, marketed as Messenger® (Eden Biosciences, Bothel, Wash.) and Vacciplant® (GOËMAR, Saint-Malo France). Examples of functional analogues include 2,6-Dicloroisonicotinic acid and Acibenzolar-S-Methyl, marketed as Actigard® (Syngenta Crop Protection, Greensboro, N.C.). Although these resistance-inducing compounds may induce PR proteins and SAR, they have a limited spectrum of uses.

The objects of the present invention are to determine the ability of a compound to induce plant resistance to pathogen attack, to use the induction of a pathogenesis-related protein to indicate the ability of a compound to increase resistance to plant disease, to use the induction of resistance to tobacco mosaic virus to determine the ability of a compound to induce resistance to other plant pathogens including fungi, bacteria, and other viruses, to identify new or novel inducers of plant disease resistance, to identify salicylates with previously unknown resistance-inducing properties, to identify novel inducers of plant disease resistance, to identify new or novel inducers of SAR and to increase the plant protective effect of SAR inducers by combining them with ROS-generating compounds.

### SUMMARY OF THE INVENTION

The present invention is directed to a method of inducing the production of PR-1a protein in plants comprising applying to said plants an effective amount of a salicylate or 3-hydroxypicolinic acid.

The present invention is also directed to a method of increasing the resistance of plants to tobacco mosaic virus comprising applying to said plants an effective amount of a salicylate or 3-hydroxypicolinic acid.

The present invention is further directed to a method of potentiating the plant protective activity of PPO inhibiting herbicides comprising applying a salicylate with a PPO inhibiting herbicide to a plant.

In a further embodiment, the present invention is directed to a method of increasing the plant protective effect of SAR inducers, preferably salicylates, comprising applying to said plants an SAR inducer and an ROS-generating herbicide.

In yet another embodiment, the present invention is directed to a systemic acquired resistance composition consisting of 4-aminosalicylic acid, 3-methoxysalicylic acid, 4-methoxysalicylic acid, 5-methoxysalicylic acid, 6-methoxysalicylic acid, 3-fluorosalicylic acid, 4-fluorosalicylic acid, 5-fluorosalicylic acid, 6-fluorosalicylic acid, 3,5-difluorosalicylic acid, 3-chlorosalicylic acid, 3,5,6-trichlorosalicylic acid, 3-fluoro-5-chlorosalicylic acid, 3-chloro-5-fluorosalicylic acid, 3,5-dichloro-6-hydroxysalicylic acid, and 3-hydroxypicolinic acid or any combination contain these compounds and a carrier.

### DETAILED DESCRIPTION OF THE INVENTION

A salicylate is defined as any substituted or otherwise unsubstituted benzoic acid having a hydroxyl group in the 2- or ortho-position, or a biologically acceptable salt or biological or chemical precursor thereof. Substitution on the benzoic acid includes mono- di-, tri- or tetra-substitution in the 3-, 4-, 5- and/or 6-positions: substituents may be chosen in any combination from: lower alkyl groups of 1 to 4 carbons; an alkyl bridge containing 3 or 4 carbons attached to the benzoic acid at two adjacent points; lower alkoxy groups of from 1 to 4 carbons; the halogens fluorine, chlorine, bromine or iodine; an amino group, wherein the nitrogen may carry 0, 1, or 2 identical or different lower alkyl groups of from 1 to 4 carbons each; the nitro group; the formyl group; the acetyl group; the hydroxymethyl group; the methoxycarbonyl group; the carboxamido or sulfonamido groups wherein the nitrogen may carry 0, 1 or 2 identical or different lower alkyl substituents of from 1 to 4 carbons each; the cyano group; an alkylthio-, alkylsulfoxy or alkylsulfonyl group, wherein the alkyl group is comprised of from 1 to 4 carbons, or a mono-, di- or trifluoromethyl group. Biologically acceptable salts include those of the common alkali metals sodium and potassium, the alkaline earths magnesium or calcium, zinc, or ammonium or simple alkylammonium cations such as mono-, di-, tri- or tetramethylammonium or other ammonium cations bearing up to 7 carbons. Biological or chemical precursors of 2-hydroxylated benzoic acid include non-hydroxylated benzoic acid and derivatives thereof having at least one ortho-position free, wherein the hydroxyl group is introduced biologically by the natural metabolic processes of the plant to which it is applied. Biological or chemical precursors of 2-hydroxylated benzoic acid also include benzoic acid compounds wherein the hydroxyl group in the 2-position is masked chemically in such a way that the masking group is labile and is easily removed once the compound has been applied to a plant, either by an enzymatic process of the plant's normal metabolism or by slow spontaneous hydrolysis. Examples of such masking groups include esters with monocarboxylic acids of from 1 to 7 carbons and trialkylsilyl ethers containing from 3 to 13 carbons.

An SAR inducer is defined as any compound that promotes resistance in a plant to a disease-causing agent, which include, but are not limited to a virus, a bacterium, a fungus, or combinations of these agents. A component of the resistance response of plants to pathogens is the induction of pathogenesis-related proteins. In addition, an SAR inducer may induce resistance to insect feeding in a plant, as defined by Enyedi et al. (1992; Cell 70: 879-886). Exemplary SAR inducers cover many structural families of compounds, but are united by their ability to induce pathogenesis-related proteins, induce resistance to plant diseases and/or pest feeding. One class of SAR inducers is the salicylates. Another class of SAR inducers includes benzothiadiazole derivatives, such as Acibenzolar-S-methyl, sold as Actigard®. Yet another example of an SAR inducer is 2,6-dichloroisonicotinic acid. Elicitors comprise another class of experimental SAR inducers that may have utility for this use.

Particularly preferred SAR inducers include 4-aminosalicylic acid, 3-methoxysalicylic acid, 4-methoxysalicylic acid, 5-methoxysalicylic acid, 6-methoxysalicylic acid, 3-fluorosalicylic acid, 4-fluorosalicylic acid, 5-fluorosalicylic acid, 6-fluorosalicylic acid, 3,5-difluorosalicylic acid, 3-chlorosalicylic acid, 3,5,6-trichlorosalicylic acid, 3-fluoro-5-chlorosalicylic acid, 3-chloro-5-fluorosalicylic acid, 3,5-dichloro-6-hydroxysalicylic acid, and 3-hydroxypicolinic acid or any combination contain these compounds.

ROS generating herbicides may be defined as inhibitors of photosystem I (paraquat or diquat).

In one embodiment of the present invention, the compositions useful in accordance with the present invention include from ______% to ______% SAR inducer and from ______% to ______% salicylate, and most preferably from ______% to ______% SAR inducer and from ______% to ______% salicylate.

In another embodiment of the present invention, the compositions include from ______% to ______% SAR inducer and from ______% to ______% ROS generating herbicide, preferably from ______% to ______% SAR inducer and from ______% to ______% ROS generating herbicide and most preferably from ______% to ______% SAR inducer and from ______% to ______% ROS generating herbicide.

The compositions are dispersed or dissolved in water to a concentration of from 15% to 0.0015%, preferably 5.0% to 0.002% and most preferably 1.0% to 0.05% for application.

The compositions may also be formulated as concentrates which are sufficiently storage stable for commercial use and which are diluted with water before use. Alternatively, each component may be formulated as a separate concentrate for mixing and dilution prior to use.

Compositions of the present invention include liquid compositions, which are ready for immediate use, and solid or liquid concentrated compositions, which require dilution before use, usually with water.

The solid compositions may be in the form of granules or dusting powders wherein the active ingredient is mixed with a finely divided solid diluent (e.g. kaolin, bentonite, kieselguhr, dolomite, calcium carbonate, talc, powdered magnesia, Fuller's earth or gypsum). They may also be in the form of dispersible powders or grains, comprising a wetting agent to facilitate the dispersion of the powder or grains in liquid. Solid compositions in the form of a powder may be applied as foliar dusts.

Liquid compositions may comprise a solution, suspension or dispersion of the active ingredients in water optionally containing a surface-active agent, or may comprise a solution or dispersion of the active ingredient in a water-immiscible organic solvent which is dispersed as droplets in water. Preferred active ingredients of the composition of the present invention are water-soluble herbicides or are readily suspended in water and it is preferred to use aqueous compositions and concentrates. The composition of the present invention may contain additional surface active agents, including for example surface active agents to increase the compatibility or stability of concentrated compositions as discussed above. Such surface-active agents may be of the cationic, anionic, or non-ionic or amphoteric type or mixtures thereof. The cationic agents are, for example, quaternary ammonium compounds (e.g., cetyltrimethylammonium bromide). Suitable anionic agents are soaps, salts of aliphatic mono esters of sulphuric acid, for example sodium lauryl sulphate; and salts of sulphonated aromatic compounds, for example sodium dodecylbenzenesulphonate, sodium, calcium, and ammonium lignosulphonate, butyinaphthalene sulphonate and a mixture of the sodium salts of diisopropyl and triisopropylnaphthalenesulphonic acid. Suitable non-ionic agents are the condensation products of ethylene oxide with fatty alcohols such as oleyl alcohol and cetyl alcohol, or with alkylphenols such as octyl- or nonyl-phenol or octylcresol. Other non-ionic agents are the partial esters derived from long chain fatty acids and hexitol anhydrides, for example sorbitan monolaurate; the condensation products of the partial ester with ethylene oxide; the lecithins; and silicone surface active agents (water soluble of dispersible surface active agents having a skeleton which comprises a siloxane chain e.g. Silwet L77®). A suitable mixture in mineral oil is ATPLUS 411 F®.

Other adjuvants commonly utilized in agricultural compositions include compatibilizing agents, antifoam agents, sequestering agents, neutralizing agents and buffers, corrosion inhibitors, dyes, odorants, spreading agents, penetration aids, sticking agents, dispersing agents, thickening agents, freezing point depressants, antimicrobial agents, and the like. The compositions may also contain other compatible components, for example, other herbicides, plant growth regulants, fungicides, insecticides, and the like and can be formulated with liquid fertilizers or solid, particulate fertilizer carriers such as ammonium nitrate, urea, and the like.

The rate of application of the composition of the present invention will depend on a number of factors including, for example, the active ingredients, the plant species whose growth is to be inhibited, the growth stage and density of the weed species, the formulation and the method of application, as for example, spraying, addition to irrigation water or other conventional means. As a general guide, however, the application rate is from 1000 to 10 liters of diluted spray solution per hectare, preferably from 200 to 100 liters per hectare.

The present invention may be illustrated by the following representative examples.

Procedures:

Plant Growth Conditions:

Tobacco (Nicotiana tabacum) plants (Xanthi nc) were grown in Promix PGX at 25 C and a 16/8 h photoperiod. Plants were fed a 1 g/L concentration of 20-20-20 (N-P-K fertilizer) twice a week. The Xanthi-nc variety carries the N-gene for resistance to TMV. Plants were treated with chemicals and/or virus at 5-6 weeks after sowing, when they had 5-7 fully-expanded leaves.

Induction and Quantification of PR-1a

For evaluating pathogenesis-related protein induction, test compounds were dissolved in ethanol and dispersed in water (0.1% v/v ethanol). The most recently fully-expanded leaf on each plant was selected for infiltration. Infiltration was accomplished using a 5 mL disposable syringe (without needle). The solution was forced through the stomates of the abaxial surface of the leaf. Treatment solution was infiltrated into the leaf until the leaf was saturated.

Proteins were harvested by the method of Yalpani et al. (1991; Plant Cell. 3:809-818). All harvests were performed 96 hours after infiltration unless indicated. Briefly, treated leaves were de-veined, the remaining tissue was cut into 1 cm×1 cm pieces and vacuum-infiltrated with ice-cold extraction buffer. Leaf tissue was blotted to remove excess buffer, and placed in a spin column assembly (Yalpani et al., 1991). The extracellular fluid containing the acidic PR-1a protein was collected by centrifugation at 1000 g for 10 minutes at 4° C. After collection, samples were stored at −80° C. Protein concentrations of extracellular fluids were determined by Bradford dye reagent (BioRad, Richmond, Calif.) used BSA as a standard. Eight to ten μg of each protein sample were analyzed by electrophoresis on 14% Native Polyacrylamide gels (14% pre-cast Tris-Glycine gels; Invitrogen, Carlsbad, Calif.). Gels were stained with colloidal blue staining reagent (Invitrogen), and PR-1a protein was quantitated using a Model GS-710 densitometer (BioRad).

Resistance Measurement

Spray Applications:

Test compounds were dissolved in ETOH and dispersed in water (0.1% v/v ethanol). Spray solutions were amended with 0.25% crop oil concentrate (v/v). Plants were sprayed to drip, and moved to a glasshouse.

TMV Inoculation and Measurement:

After spraying, one leaf per plant was inoculated with 1.0 ug TMV (U1; vulgare strain) at the times indicated. Six days post-inoculation, leaves were removed and the diameters of 20 necrotic lesions were measured per leaf. The mean lesion diameters from at least four plants per treatment were measured. Lesion diameter measures were subjected to two way analysis of variance (ANOVA) and Duncan's multiple range procedure at p=0.05.

### EXAMPLE 1

Substituted salicylates were tested to determine their ability to induce PR-1a protein. Included in this list are salicylates known to be SAR inducers according to Conrath et al. (1995. PNAS USA 92:7143). Table 1 lists the PR-1a induction by mono-substituted salicylates.

The hydroxyl group in the 2 position, ortho to the carboxylic acid functionality, is necessary for PR induction. Benzoic acid was unable to induce PR-1a accumulation, while substitution with either fluorine (2-Fluorobenzoic acid) or a thiol group (Thiosalicylic acid) significantly reduced activity (Table 1).

TABLE 1
Pathogenesis-related protein induced by
salicylates with one substitution

| Position | Substituent | Name | Mean PR Induction¹ |
|---|---|---|---|
| 1 | CONH2 | Salicylamide | 0 |
| | CONHOH | Salicylhydroxamic acid | |
| 2 | H | Benzoic acid | 0 |
| | F | 2-Fluorobenzoic acid | 0.06 |
| | SH | Thiosalicylic acid | 0.59 |
| | *Acetyl | Acetylsalicylic acid | 1.68 |
| 3 | NH2 | 3-Aminosalicylic acid | 0.54 |
| | OH | 3-Hydroxylsalicylic acid | 0 |
| | OCH3 | 3-Methoxysalicylic acid | 0.49 |
| | CH3 | 3-Methylsalicylic acid | 0.22 |
| | NO2 | 3-Nitrosalicylic acid | 0.27 |
| | F | 3-Fluorosalicylic acid | 2.13 |
| | Cl | 3-Chlorosalicylic acid | 2.67 |
| | CHO | 3-Formylsalicylic acid | 0.06 |
| | i-Pr | 3-Isopropylsalicylic acid | 0 |
| | Phe | 3-Phenylsalicylic acid | 0 |
| 4 | *NH2 | 4-Aminosalicylic acid | 0.07 |
| | OH | 4-Hydroxylsalicylic acid | 0 |
| | OCH3 | 4-Methoxysalicylic acid | 0.17 |
| | CH3 | 4-Methylsalicylic acid | 0.05 |
| | F | 4-Fluorosalicylic acid | 0.83 |
| | *Cl | 4-Chlorosalicylic acid | 0.32 |
| 5 | NH2 | 5-Aminosalicylic acid | 0 |
| | OH | 5-Hydroxylsalicylic acid | 0 |
| | OCH3 | 5-Methoxysalicylic acid | 0.12 |
| | CH3 | 5-Methylsalicylic acid | 0 |
| | NO2 | 5-Nitrosalicylic acid | 0.15 |
| | F | 5-Fluorosalicylic acid | 1.60 |
| | *Cl | 5-Chlorosalicylic acid | 1.07 |
| | Br | 5-Bromosalicylic acid | 0.41 |
| | CHO | 5-Formylsalicylic acid | 0 |
| | I | 5-Iodosalicylic acid | 0 |
| | CN | 5-Cyanosalicylic acid | 0.85 |
| 6 | OH | 6-Hydroxylsalicylic acid | 0.86 |
| | OCH3 | 6-Methoxysalicylic acid | 0.31 |
| | CH3 | 6-Methylsalicylic acid | 0.16 |
| | F | 6-Fluorosalicylic acid | 0.29 |

¹Relative induction of PR-1a protein at 96 h post infiltration as compared to 1 mM SA, which equals 1.
*Known SAR inducer (Conrath et al., 1995. PNAS USA 92: 7143).

The PR-1a inducing activity of salicylates with multiple substitutions is shown in Table 2. Of these compounds, only one, 3,5-Difluorosalicylic acid is a known inducer of SAR. Salicylates with two substitutions, particularly ortho (3) and para (5) to the hydroxyl group, had the greatest effect on the PR-1a inducing activity. Substitution with a nitro group, which donates electrons to the saturated ring, decreased activity (see 3-Nitrosalicylic acid and 5-Nitrosalicylic acid in Table 1), while the 3,5-Dinitrosalicylic acid is completely deactivated. In contrast, halogens are electron withdrawing, and their substitution in the 3 and 5 positions may increase PR-1a inducing activity. For halogen substitutions in the 5 position (para to the hydroxyl) the order of activity is F>Cl>Br>I. For the 3,5-Dihalogen salicylates, the order of PR-1a inducing activity is the same as for the 5-substituted: F>Cl>Br. The 3,5-Dihalogen salicylates substituted with a mix of the chlorine and fluorine induce PR-1a less so than either the difluoro or dichloro salicylate. At least one contributing factor to this may be the molecular radius of the halogen, the fluorine being the smallest, followed by chlorine, bromine, and finally iodine.

TABLE 2
Pathogenesis-related protein 1a induced by
salicylates with multiple substitutions

| Compound | Mean PR Induction¹ |
|---|---|
| 3,5-Dinitrosalicylic acid | 0 |
| 3,5-Difluorosalicylic acid | 3.83 |
| *3,5-Dichlorosalicylic acid | 1.81 |
| 3,5-Dibromosalicylic acid | 0.25 |
| 3,5,6-Trichlorosalicylic acid | 0.25 |
| 3-Fluoro-5-chlorosalicylic acid | 1.46 |
| 3-Chloro-5-fluorosalicylic acid | 1.70 |
| 3,5-Dichloro-6-hydroxysalicylic acid | 1.41 |
| 3-Methoxy-5-chlorosalicylic acid | 0.15 |
| 2,6-Difluorobenzoic acid | 0 |
| 2,3,6-Trifluorobenzoic acid | 0.02 |

¹Relative induction of PR-1a protein at 96 h post infiltration of 1 mM of the tested compounds as compared to 1 mM SA, which equals 1.
*Known SAR inducers (Conrath et al., 1995. PNAS USA 92: 7143).

A time course for the induction of PR-1a protein is shown in Table 3. Treatment with the fluorinated salicylates, 5-Fluorosalicylic acid and 3,5-Difluorosalicylic acid, resulted in more induction at 96 hours than the corresponding chlorinated salicylates. At 192 h after treatment, 3,5-Dichlorosalicylic acid showed the greatest induction of PR-1a.

TABLE 3
Induction of tobacco PR-1a protein by salicylates

PR-1a protein
following treatment¹

| Treatment | 96 h | 192 h |
|---|---|---|
| Control | 0 | 0 |
| Salicylic acid, 1 mM | 1.00 | 1.46 |
| *5-Chlorosalicylic acid, 1 mM | 1.02 | 1.75 |
| 5-Fluorosalicylic acid, 1 mM | 1.30 | 1.74 |
| *3,5-Dichlorosalicylic acid, 1 mM | 1.83 | 4.66 |
| 3,5-Difluorosalicylic acid, 1 mM | 3.64 | 3.21 |

¹Relative induction of PR-1a protein at 96 h post infiltration of 1 mM of the tested compounds as compared to 1 mM SA, which equals 1.
*Known SAR inducers (Conrath et al., 1995. PNAS USA 92: 7143).

### EXAMPLE 2

Induction of PR-1a protein also allowed us to identify SAR activity in derivatives of picolinic acid (Table 4). Of the picolinic acid derivatives tested, only 3-Hydroxypicolinic acid induced PR-1a accumulation more than equimolar SA.

TABLE 4
PR protein induction by 1 mM Picolinic acid derivatives

| Compound | Mean PR Protein Induction¹ |
|---|---|
| Picolinic Acid | 0 |
| 3-Hydroxypicolinic acid | 1.56 |
| Picolinic acid N-Oxide | 0.51 |

¹Relative induction of PR-1a protein at 96 h post infiltration of 1 mM of the tested compounds as compared to 1 mM SA, which equals 1.

### EXAMPLE 3

Although PR protein accumulation is one of the features of SAR, the relationship between these is not necessarily causal. For the salicylate derivatives and tested, we found a correlative relationship (R²=0.53) between PR protein accumulation, and reduction in TMV lesion diameter, a measure of SAR. The correlative relationship between confirms the use of PR-1a as a screening tool for SAR activity

TABLE 5
Relation between induction of pathogenesis-related protein
and decrease in TMV lesion diameter.

| | Mean PR | Percent decrease |
|---|---|---|
| | Protein | in TMV-lesion |
| Compound | Induction¹ | diameter² |
| Control | 0 | 0 |
| Salicylic acid/Sodiumsalicylate | 1.0 | 30.1 |
| 4-Aminosalicylic acid | 0.08 | 22.9 |
| 3-Methoxysalicylic acid | 0.49 | 8.9 |
| 4-Methoxysalicylic acid | 0.17 | 17.6 |
| 5-Methoxysalicylic acid | 0.12 | 9.7 |
| 6-Methoxysalicylic acid | 0.31 | 13.3 |
| 3-Fluorosalicylic acid | 2.13 | 34.4 |
| 4-Fluorosalicylic acid | 0.83 | 36.2 |
| 5-Fluorosalicylic acid | 1.60 | 38.0 |
| 6-Fluorosalicylic acid | 0.29 | 24.3 |
| 3,5-Difluorosalicylic acid | 3.83 | 55.0 |
| 3-Chlorosalicylic acid | 2.67 | 55.1 |
| *4-Chlorosalicylic acid | 0.32 | 30.2 |
| *5-Chlorosalicylic acid | 1.07 | 38.7 |
| *3,5-Dichlorosalicylic acid | 1.81 | 36.6 |
| 3,5,6-Trichlorosalicylic acid | 0.25 | 45.1 |
| 3-Fluoro-5-chlorosalicylic acid | 1.70 | 47.4 |
| 3-Chloro-5-fluorosalicylic acid | 1.70 | 34 |
| 3,5-Dichloro-6-hydroxysalicylic acid | 1.41 | 48.3 |

¹Relative induction of PR-1a protein at 96 h post infiltration of 1 mM of the tested compounds as compared to 1 mM SA, which equals 1.
²Percent reduction in the diameter of TMV lesions.
*Known SAR inducers (Conrath et al., 1995. PNAS USA 92: 7143).

### EXAMPLE 4

Measuring a decrease in TMV lesion diameter following spray application provides a measure of SAR. A study showing the effect of treatment time before TMV exposure is presented in Table 6. When tobacco was sprayed with 3,5-Difluorosalicylic acid 96 hours before TMV treatment, its ability to reduce TMV-lesion size was statistically equal to that induced by 5 mM Salicylic acid.

TABLE 6
Application of salicylates induces resistance of Xanthi-nc
tobacco to tobacco mosaic virus (TMV)

Lesion diameter (mm)

| Treatment | 48 h* | 96 h* |
|---|---|---|
| Control | 2.82 D | 3.64 C |
| Salicylic Acid 1 mM | 2.20 BC | 2.52 B |
| Salicylic Acid 5 mM | 1.88 A | 1.68 A |
| 3,5-Difluorosalicylic acid, 1 mM | 2.04 AB | 1.85 A |

n = 5 plants. Mean separation by Duncan's New Multiple Range (α = 0.05). Means followed by the same letter are not statistically different.
*Hours spray application hours preceded TMV inoculation.

### EXAMPLE 5

Among the compounds capable of producing reactive oxygen species (ROS) in plants, herbicides are the most commonly used. The protoporphyrinogen oxidase (PPO) inhibitor herbicides, particularly the diphenyl ethers, are thought to work through the generation of ROS and are known inducers of plant resistance to disease (Haddad et al. 2001. World Intellectual Property Organization WO 01/58268 A1). We tested the combination sodium salicylate with the PPO inhibitor lactofen, and found that the combination caused a synergistic increase in the induction of PR-1a in tobacco (Table 7).

TABLE 7
Induction of PR-1a in Xanthi-nc tobacco by sodium salicylate (NaSA)
or Cobra 2EC (Lactofen; 1-(carboxyethoxy) ethyl 5-[2-chloro-4-
(tri-fluoromethyl)phenoxy]-2-nitrobenzoate) treatments¹.

| Treatment | PR-1a² |
|---|---|
| Control | 0 |
| 1600 ppm NaSA | 1.00 |
| 8000 ppm NaSA | 4.56 |
| 290 ppm Lactofen | 0.44 |
| 1450 ppm Lactofen | 2.00 |
| 290 ppm Lactofen + 1600 ppm SA | 9.00 |

¹PR protein harvested 96 hours after spray application.
²Relative induction of PR-1a protein as compared to 1 mM SA, which equals 1.

The bipyridilium herbicides are inhibitors of photosystem I. They include paraquat, which is a commonly-used ROS generator. In combination with sodium salicylate, which protects the plants from paraquat damage, increased accumulation of PR-1a protein was observed (Table 8). Moreover, the combination of paraquat and NaSA increased the resistance of plants to TMV greater than that seen with salicylate alone.

TABLE 8
Induction of PR-1a and resistance to TMV in Xanthi-nc
tobacco by sodium salicylate (NaSA) or methyl
viologen (Paraquat;) treatments¹.

| Treatment | PR-1a¹ | Lesion Diameter (mm)² |
|---|---|---|
| 48 hours after Application | | |
| Control | 0 | 2.79 C |
| NaSA (1600 mg/l)SA | 0.34 | 1.17 B |
| NaSA (1600 mg/l) + Paraquat | 0.719 | 0.90 A |
| (150 mg/l) SA + MV | | |
| 96 hours after Application | | |
| Control | 0 | 2.37 C |
| NaSA (1600 mg/l)SA | 1.02 | 1.75 AB |
| NaSA (1600 mg/l) + Paraquat | 2.19 | 1.63 AB |
| (150 mg/l) SA + MV | | |

¹Relative induction of PR-1a protein of tested compounds as compared to infiltration with 1 mM SA at 96 h, which equals 1.
²n = 4 plants. Mean separation by Duncan's New Multiple Range (α = 0.10) at each time point.
Means followed by the same letter are not statistically different.

1. A method of inducing the production of PR-1a protein in plants comprising applying to said plants an effective amount of a salicylate or 3-hydroxypicolinic acid.

2. A method of increasing the resistance of plants to tobacco mosaic virus comprising applying to said plants an effective amount of a salicylate or 3-hydroxypicolinic acid.

3. A method of potentiating the plant protective activity of SAR inducers comprising applying a salicylate with an SAR inducer to a plant.

4. A method of potentiating the plant protective activity of PPO inhibiting herbicides comprising applying a salicylate with a PPO inhibiting herbicide to a plant.

5. A method of increasing the plant protective effect of SAR inducers, comprising applying to said plants an SAR inducer and an ROS-generating herbicide.

6. The method of claim 5 where the SAR inducer is a salicylate.

7. A systemic acquired resistance composition consisting of 4-aminosalicylic acid, 3-methoxysalicylic acid, 4-methoxysalicylic acid, 5-methoxysalicylic acid, 6-methoxysalicylic acid, 3-fluorosalicylic acid, 4-fluorosalicylic acid, 5-fluorosalicylic acid, 6-fluorosalicylic acid, 3,5-difluorosalicylic acid, 3-chlorosalicylic acid, 3,5,6-trichlorosalicylic acid, 3-fluoro-5-chlorosalicylic acid, 3-chloro-5-fluorosalicylic acid, 3,5-dichloro-6-hydroxysalicylic acid, and 3-hydroxypicolinic acid or any combination contain these compounds and a carrier.
